(19) United States
(12) Plant Patent Application Publication
(10) Pub. No.: US 2003/0018999 P1
(43) Pub. Date: Jan. 23, 2003

(54) PLUM TREE NAMED 'EARLY DANAE'

(76) Inventor: George Flagler, Parlier, CA (US)

Correspondence Address: MARK D MILLER, KIMBLE, MACMICHAEL & UPTON, 5260 NORTH PALM AVENUE, SUITE 221, FRESNO, CA, 93704, US

(21) Appl. No.: 09/908,876

(22) Filed: Jul. 18, 2001

(51) Int. Cl. A01H 5/00

(52) U.S. Cl. PLT/184

(57) ABSTRACT

A new variety of plum tree which is remotely similar to the unpatented ‘Early Beaut’ plum tree but is distinguished from it by producing uniformly purple skin coloration fruit which are mature for harvesting and shipment one week earlier than the ‘Early Beaut’ plum tree and exhibits superior holding capacity than the ‘Early Beaut’ plum tree and having more yellow flesh throughout.

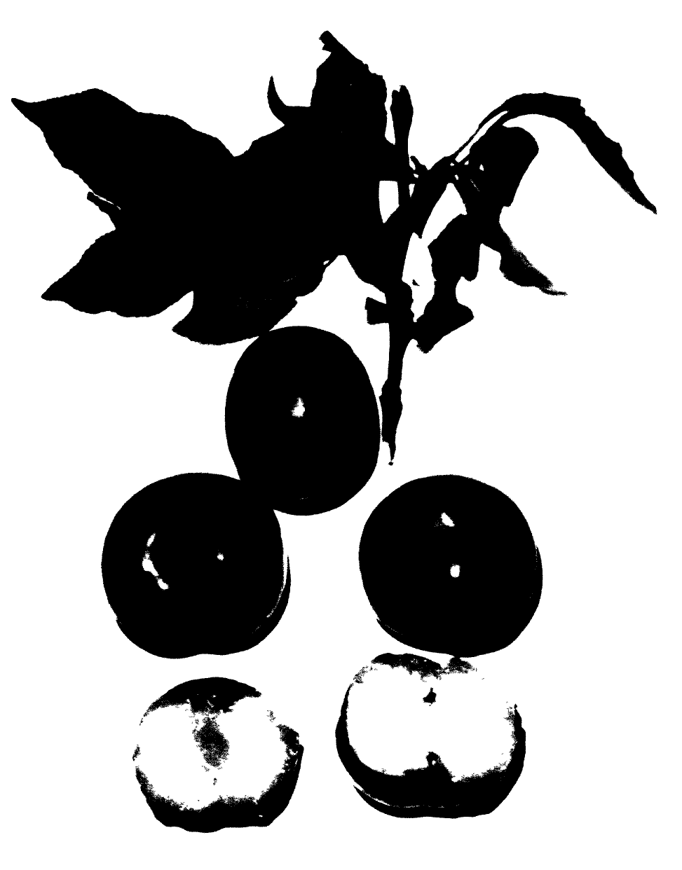

### BACKGROUND OF THE NEW VARIETY

This present invention relates to a new and distinct variety of plum tree which will hereinafter be denominated varietally as the ‘Early Danae’ plum tree and more particular to a plum tree which produces fruit which are mature for commercial harvesting and shipment about May 20-25 in the San Joaquin Valley of Central California and which is further distinguished from one of its parent ‘Showtime’ plum tree (U.S. Plant Pat. No. 8037) in that it produces fruits that have a dark purple skin coloration which can be retained on the tree and remains firm to a high level of maturity. In addition, the flesh of the fruit of this invention is a yellow gold color throughout with the shape of the fruit being somewhat flatter at the stem end.

### ORIGIN AND ASEXUAL REPRODUCTION OF THE NEW VARIETY

The present variety was discovered as an open, pollinated hybrid of the ‘Showtime’ plum tree (U.S. Plant Pat. No. 8036) in a sixteen acre plum orchard located approximately ½ mile south of Manning Avenue in Parlier, Calif. The grower has observed this invention since 1999 and confirmed that distinctive characteristics of the parent tree are precisely reproduced in the progeny.

### SUMMARY OF THE NEW VARIETY

The ‘Early Danae’ plum tree is characterized as to novelty by producing a medium size fruit generally of dark purple skin coloration, ripe for commercial harvest and shipment approximately May 20-25 in the San Joaquin valley of Central California. The new and novel variety is an open pollinated hybrid of the ‘Showtime’ plum tree (U.S. Plant Pat. No. 8037) from which it is distinguished principally by producing a uniformly dark purple skin fruit of medium size and having generally a yellow gold flesh coloration throughout with fruit shape more globular with a somewhat flattened stem end, ripe for commercial harvest and shipment approximately three to four weeks before the ‘Showtime’ plum tree fruit.

### BRIEF DESCRIPTION OF THE DRAWING

The accompanying drawing is a color photograph showing mature fruit of the new variety of plum tree of the present invention, including fruit showing just the ventral side of the fruit. The second plan shows the stem end (bottom view of the fruit). The third plan shows the apex end. The fourth plan shows the fruit sectioned along the suture plant with the stone removed exposing the flesh and the pit in the cavity. The final plan shows the foliage and twig of the new variety displaying the upper and lower surface of the leaves.

### DETAILED DESCRIPTION

Referring to more specific pomological details of this new and distinct variety of plum tree, the following has been observed under the ecological conditions prevailing at the orchard of origin which is located near Parlier in the San Joaquin Valley of Central California. All major color code designations are by reference to the Dictionary of Color by Maerz & Paul, First Edition 1930. Common color names are also occasionally used.

### TREE

Size: Medium, similar in size and growth habit to ‘Showtime’ (U.S. Plant Pat. No. 8036)—typical size for plum trees

Vigor: Good at 3^{rd} year of growth

Figure (Form):Upright and spreading with open vase system of training Productivity is good to very good for tree in 3^{rd} year of growth. Regularity of bearing appears to be regular (i.e. every year, not alternate years)

Trunk Size: Stocky—Diameter 20 inches, 18 inches above soil level, somewhat shaggy Color Code—Sandy beige (14-A-3)

Lenticels.—Oval form, small and short Color Code—Brown Number—Numerous.

Branches:

Size.—Medium—9 inches in diameter, 40 inches above ground; angle 35-38° Surface Texture Mature—Somewhat shaggy Immature—Smooth.

Color code (one year of older).—Cattail café noir (8-H-12).

Color code (immature).—Parrot green (21-L-6).

### LEAVES

Size: Medium

Length: 95-105 mm (3.74-4.13 inches)

Width: 35-40 mm (1.38-1.57 inches)

Shape: Lanceolate

Texture: Smooth

Color Code:

Upwardly disposed surface (upper side).—Hunter green (24-C-11).

Downwardly disposed surface (underside).—Near civette green (22-L-8).

Marginal form.—Double serrated.

Leaf Vein:

Color code.—Kildare green (19-I-5).

Thickness: 1 mm (0.039 inches)

Glandular Characteristics: Globular Color Code—Green Size—Small Number—Two

Petiole:

Size.—Medium.

Length.—12-15 mm (0.472-0.590 inches)

Diameter.—2 mm (0.078 inches)

Color code.—Kildare green (19-I-5)

### FLOWERS

Flower Buds: Hardy under ecological conditions in the San Joaquin Valley

Size: 5 mm (0.193 inches) long, 3 mm (0.118) wide

Form: Conic

Bud Scales: Color Code-Green

Generally: Showy

Date of Bloom: 10% as of February 25-26, 40% bloom as of February 28-30. It is not comparable to parent.

Size: Small

Diameter:

Fully expanded.—20-22 mm (0.787-0.866 inches).

Bloom Quality: Abundant

Fragrance: None

Petals:

Size.—Small, 5 in number. Length—7-9 mm (0.276-0.350 inches) Width—8 mm (0.315 inches)

Form.—Broadly ovate.

Color code.—White.

Petal claws.—Broadly truncate. Length—less than 1 mm (0.039 inches) Width—1 mm (0.039 inches)

Petal margins.—Round with undulated margins.

Flower pedicel.—Very short, 1 mm (0.039 inches). Color Code—Green Surface—Slightly pubescent Diameter—2 mm (0.079 inches)

Sepals:

Surface.—Pubescent.

Size.—Medium to large.

Form.—Broadly ovate.

Color code.—Green.

Number.—Five.

Calyx:

Color code.—Greenish brown at base.

Anthers:

Size.—Small.

Color code.—Sea green (11-G-7).

Stamen: Very small, less than 1 mm (0.039 inches)

Number.—22-25.

Pistil:

Length.—Small on average.

Number.—1

Color code.—Yellow gold (18-I-5).

Surface.—Pubescent.

### FRUIT

Date of Maturity: May 20-25 in a normal year

Size: Medium to Large

Diameter axial plane.—54-65 mm (2.126-2.558 inches).

Transverse in suture plane.—48-57 mm (1.89-2.244 inches)

Transverse at right angle to suture plane.—41-45 mm (1.64-1.773 inches).

Form: Uniform, symmetrical

Suture: Shallow and inconspicuous, but occurs from apex to stem end of fruit

Ventral Surface: Smooth

Stem Cavity:

Shape.—Generally acute.

Width.—10-12 mm (0.394-0.472 inches).

Depth.—12 mm (0.472 inches).

Length.—16-18 mm (0.63-0.709 inches).

Stem Length: 10 mm (0.394 inches)

Apex: Shaped slightly indented

Pistil Point: Variable

Skin: Thin for plums

Texture: Medium

Tendency to Crack: None observed

Color Codes:

Blush color.—None observed, since this fruit is a dark color.

Ground color.—Burgundy (56-E-8).

Flesh color.—Empire (9-K-3).

Color at surface of pit cavity.—Sunflower dandelion (9-L-4).

Color of pit well.—Sunflower dandelion (9-L-4).

Juice Production: Juicy

Flavor: Very good

Aroma: Moderate

Texture: Medium firm, meaty

Fibers:

Number.—Few.

Texture.—Firm.

Ripening: Even

Eating Quality: Very good

Stone:

Attachment.—Semi-cling.

Fibers.—None to few.

Size.—Short. Length—18 mm (0.709 inches) Width—19 mm (0.748 inches) Diameter—9 mm (0.354 inches)

Form: Generally oval

Apex: Round with small point

Color Code:

When dry.—Near capucine buff (9-G-5).

Bases: Slightly rounded

Sides: Generally equal

Texture: Grooves on both sides of the pit

Ridges.—Dorsal edge smooth with ventral edge winged from base to apical.

Tendency to split.—None known.

Use.—Fresh market.

Quality:

Ripening quality.—Good

Resistance to disease.—Unknown as of this time.

Shipping and handling qualities.—Good, with winter hardiness of the plum tree; not susceptible to damage during dormant season and no evidence of heat damage during the growing season

The above description of this new variety of plum tree is based on the growing conditions prevalent near Parlier, California, in the Central San Joaquin Valley of California; variations of the usual magnitude, such as differences in maturity date and production, may be due to cultural practices including irrigation, fertilization, pruning, fruit thinning and primary climate changes. Typical production for a single tree during year 2000 was seven 28-pound cartons.

1. A new and distinct variety of plum tree as described and illustrated which is remotely similar to the unpatented ‘early beaut’ plum tree but is distinguished from it by producing uniformly purple skin coloration fruit which are mature for harvesting and shipment one week earlier than the ‘early beaut’ plum tree and exhibits superior holding capacity than the ‘early beaut’ plum tree and having more yellow flesh throughout.
